SEQUENCE LISTING (6 sequences)

SEQ ID NO: 1 - protein, 39 aa, Lactobacillus paracasei
  source 1..39: note = GM-080
MELLNEKELAHVIGGKRKCPKTPFDNTPGAWFAHLILGC

SEQ ID NO: 2 - protein, 45 aa, Lactobacillus paracasei
  source 1..45: note = GM-080
MKQFDEQKMVNMSDEELLEFIGGDSIRDVSPTFNKIRRWFDGLFK

SEQ ID NO: 3 - DNA, 21 bp, synthetic construct
  source 1..21: mol_type = other DNA
ggccgtgtaggattcggataa

SEQ ID NO: 4 - DNA, 20 bp, synthetic construct
  source 1..20: mol_type = other DNA
gtggtaaacgaaagtgcccc

SEQ ID NO: 5 - DNA, 20 bp, synthetic construct
  source 1..20: mol_type = other DNA
agatgcgagcgaaaactcgt

SEQ ID NO: 6 - DNA, 20 bp, synthetic construct
  source 1..20: mol_type = other DNA
attggaggtgacagcatccg

(19) United States
(12) Patent Application Publication
(10) Pub. No.: US 2025/0197460 A1
(43) Pub. Date: Jun. 19, 2025

(54) USE OF LACTOBACILLUS PARACASEI GM-080 AND PEPTIDES THEREOF FOR ANTIMICROBIA

(71) Applicant: GenMont Biotech Incorporation, Tainan City (TW)

(72) Inventors: Wan-Hua TSAI, Kaohsiung City (TW); Yi-Ting Fang, Tainan City (TW); Chia-Hsuan Chou, Tainan City (TW); Chih-Feng Kuo, Kaohsiung City (TW); Ni-Na Tsao, Kaohsiung City (TW); Jhih-Hua Jhong, Gongguan Township (TW); Tzong-Yi Lee, Hsinchu City (TW)

(21) Appl. No.: 18/585,638

(22) Filed: Feb. 23, 2024

(30) Foreign Application Priority Data
Dec. 14, 2023 (TW) 112148733

(51) Int. Cl. C07K 14/335; A61K 38/00; A61P 31/04; C12N 1/20; C12R 1/225

(52) U.S. Cl. CPC C07K 14/335; A61P 31/04; C12N 1/205; A61K 38/00; C12R 2001/225

(57) ABSTRACT

A use of Lactobacillus paracasei GM-080 and peptides thereof for preparing an antimicrobial composition. The composition is a pharmaceutical composition, a nutritional supplement, or a health care food, wherein the deposit number of Lactobacillus paracasei GM-080 is BCRC 910220 or CCTCC M 204012. In addition, a peptide of Lactobacillus paracasei GM-080 and compositions thereof are provided.

### REFERENCE TO RELATED APPLICATIONS

The present application is based on, and claims priority from, Taiwan application number 112148733, filed Dec. 14, 2023, the disclosure of which is hereby incorporated by reference herein in its entirety.

### REFERENCE TO AN ELECTRONIC SEQUENCE LISTING

The contents of the electronic sequence listing (sequence listing.xml; Size: 6,403 bytes; and Date of Creation: Feb. 15, 2024) is herein incorporated by reference in its entirety.

### BACKGROUND OF THE INVENTION

Upper Respiratory Tract Infections (URTIs) are the most common diseases in the world that can be treated with antibiotics, URTIs including rhinitis, nasal congestion (including pyogenic rhinitis and nasal congestion) (about 44.4%), acute laryngopharyngitis (about 10.8%), acute otitis media (about 5.1%), effusion otitis media (about 1.2%), acute sinusitis (about 8.2%). Acute laryngopharyngitis are common at the times when autumn is changing into winter and when spring is changing into summer. Viral infection is the most common cause of acute pharyngitis. However, most of the symptoms of viral infection are relatively mild and will be self-healed.

However, the laryngopharyngitis caused by bacterial infection easily leads to serious complications. In the population of 5-15 years old, the dominant pathogenic bacteria are Streptococcus pyogenes, also known as Group A Streptococcus (GAS), which easily invades the population of 5-15 years old and may cause complications, such as rheumatic heart disease, myocarditis, polyarthritis, glomerulonephritis, septicemia and the like, all of which may be life-threatening. The main pathogenic bacteria of acute otitis media and acute sinusitis are mainly Streptococcus pneumoniae, Haemophilus influenzae and Moraxella catarrhalis.

In clinic, antibiotics are often used to alleviate the upper respiratory tract infection caused by pathogenic bacteria, but with the continuous appearance of the side effects of antibiotics and antibiotic strains clinically and because of antibiotics not capable of being used for daily health care, scientists begin to look for a method capable of enhancing mucosal immunity and inhibiting pathogenic bacteria with non-antibiotics. Among them, probiotics are regarded as a safe, effective and positive means without side effects, which can be used for preventing upper respiratory tract infection.

The mechanism for preventing respiratory tract infection by probiotics can inhibit the harm caused by pathogenic bacteria toxins, inhibit the attachment and invasion of pathogenic bacteria to host cells, and improve the mucosal immunity, in addition to directly inhibiting the growth of common pathogenic bacteria in the respiratory tract. In the past, animal and clinical research also indicates that the symptoms and frequency of upper respiratory infections caused by viruses can be alleviated by some probiotic strains.

It should be a very good and reasonable option to prevent the occurrence of upper respiratory diseases with probiotics. Among many other strains, such as S. salivarius M18, PS4, JIM8777, CCHSS3, 57.I, etc., Streptococcus salivarius K12 found in 2001 is the most famous strain for inhibiting Streptococcus pyogenes in the past. The common antimicrobial mechanism thereof is that the Streptococcus salivarius can secrete bacteriostatin, such as megaplasmid-encoded class I lantibiotics, salivaricin A2, salivaricin B, etc, thereby inhibiting the growth of Streptococcus.

The subject to be solved by the present invention is to find probiotics with antimicrobial efficacy and active ingredients therein.

### FIELD OF THE INVENTION

The present invention relates to the technical field of Lactobacillus paracasei (also known as Lacticaseibacillus paracasei) resisting to Streptococcus pyogenes, in particular to the inhibition of Streptococcus pyogenes by secreting antimicrobial peptides.

### SUMMARY OF THE INVENTION

In view of this, after many years of research, the inventors of the present invention successfully isolated probiotics capable of inhibiting Streptococcus pyogenes. Such probiotics are Lactobacillus paracasei. The probiotics and peptides thereof have the efficacy of inhibiting Streptococcus pyogenes, and thus providing a novel and safe prevention strategy for upper respiratory diseases.

In order to achieve the above objects, the present invention provides a peptide with an antimicrobial effect, and the peptide sequence is set forth in SEQ ID NO: 1.

In an embodiment of the present invention, the peptide is secreted by Lactobacillus paracasei GM-080 having a deposit number of BCRC 910220 or CCTCC M 204012.

Additionally, the present invention provides a composition with an antimicrobial effect, comprising the peptide as described above.

In an embodiment of the present invention, the composition can further comprise a pharmaceutically acceptable carrier.

In an embodiment of the present invention, the composition is in the form of a solution, a suspension, an emulsion, a powder, a pastille, a pill, a syrup, a buccal ingot, a tablet, a chewing gum, a thick slurry, or a capsule.

In an embodiment of the present invention, the composition can further comprise an edible material.

Additionally, the present invention provides a use of Lactobacillus paracasei GM-080 or peptides thereof for preparing an antimicrobial composition, the Lactobacillus paracasei having a deposit number of BCRC 910220 or CCTCC M 204012. The antimicrobial effect is intended to inhibit the growth of microorganisms. The peptide sequence is set forth in SEQ ID NO: 1.

In an embodiment of the present invention, the inhibited microorganism is Streptococcus.

In an embodiment of the present invention, the inhibited microorganism is Streptococcus pyogenes.

In an embodiment of the present invention, the composition is a pharmaceutical composition, a nutritional supplement, or a health care food.

In an embodiment of the present invention, the peptide is secreted by Lactobacillus paracasei GM-080.

The present invention provides a Lactobacillus paracasei GM-080 capable of inhibiting the growth and the attachment to laryngopharyngeal cells of Streptococcus pyogenes. Lactobacillus paracasei GM-080 is capable of colonizing the respiratory tract and effectively reducing the number of viable bacteria of local Streptococcus pyogenes. In particular, the Streptococcus pyogenes is inhibited by peptide AMP-01026 (SEQ ID NO: 1) secreted by Lactobacillus paracasei GM-080. The present invention further provides a composition comprising, as active ingredients, Lactobacillus paracasei GM-080 or peptide AMP-01026 which are capable of inhibiting the growth and the attachment to laryngopharyngeal cells of Streptococcus pyogenes. The composition provided by the present invention has the advantage of low side effects due to probiotics or peptides as active ingredients.

### BRIEF DESCRIPTION OF THE DRAWINGS

The techniques of present invention would be more understandable from the detailed description given herein below and the accompanying figures are provided for better illustration, and thus description and figures are not limitative for present invention, and wherein:

FIG. 1A is the results of the abilities to inhibit the growth of Streptococcus pyogenes FIG. 1B is the analyses of the abilities to inhibit the attachment to laryngopharyngeal cells.

FIG. 2A is an analysis of sites where GM-080 can colonize the laryngopharynx of a mouse in a viable bacteria form.

FIG. 2B is an analysis of sites where GM-080 can colonize the laryngopharynx of a mouse in a viable bacteria form.

FIG. 3 is an experimental procedure for evaluating the protection efficacy of preventing Streptococcus pyogenes from infecting an animal.

FIG. 4A is the analyses of the mitigation of the reduction in weight of mice caused by Streptococcus pyogenes by GM-080

FIG. 4B is the analyses of the mitigation of the reducing weight of mice caused by Streptococcus pyogenes by GM-080.

FIG. 4C is the mitigation of the number of Streptococcus pyogenes on the larynx in mice by GM-080.

FIG. 5A is the analyses of the DNA and amino acid sequences of antimicrobial peptides AMP-01026 and AMP-01240 of GM-080.

FIG. 5B is the analyses of differences of expression amount thereof from other Lactobacilli paracasei measured by Q-PCR.

FIG. 6A is the results of chromatographic separation of AMP-01026.

FIG. 6B is the results of mass spectrometric analysis of AMP-01026.

FIG. 7A is the results of chromatographic separation of AMP-01240.

FIG. 7B is the results of mass spectrometric analysis of AMP-01240.

FIG. 8A is analyses for evaluating the abilities of AMP-01026 to inhibit Streptococcus pyogenes in a co-culture manner.

FIG. 8B is analyses for evaluating the abilities of AMP-01240 to inhibit Streptococcus pyogenes in a co-culture manner.

FIG. 9A is an evaluation of the abilities of AMP-01026 to inhibit Streptococcus pyogenes in a dish coating manner.

FIG. 9B is an evaluation of the abilities of AMP-01240 to inhibit Streptococcus pyogenes in a dish coating manner.

### DETAILED DESCRIPTION OF THE INVENTION

All technical and scientific terms described in the specification, unless otherwise defined, are of the meanings commonly understood by one skilled in the art.

As described in the specification and claims, the singular terms “a”, “an” and “the”, unless otherwise indicated, may all refer to more than one object.

As described in the specification, “or”, “as well as”, “and”, unless otherwise indicated, may all refer to “or/and”. In addition, neither term “including” nor term “comprising” is an open-ended connection word with limitations. The preceding paragraphs are only systematic reference and should not be construed as limitations on the scope of the invention.

The compositions provided by the present invention are a dosage form applicable to the compositions of the present invention and which can be prepared with the active ingredient or composition provided in the present invention and at least one pharmaceutically acceptable vehicle by using the techniques that are well known to those skilled in the art to which the present invention belongs. Among them, the dosage forms include, but are not limited to, solutions, emulsions, suspensions, powders, pastilles, buccal ingots, tablets, chewing gums, capsules, and the like or the dosage forms applicable to the present invention.

The term “pharmaceutically acceptable” means that the substance or composition has to be compatible with the other ingredients of the pharmaceutical formulation thereof and does not exacerbate the symptoms of the patient.

The term “pharmaceutically acceptable carrier” includes one or more types of carriers selected from the group consisting of a solvent, an emulsifier, a suspending agent, a decomposing agent, an adhesive, an excipient, a stabilizing agent, a chelating agent, a diluent, a gelling agent, a preservative, a lubricant, a surfactant, and the like or the carriers applicable to the present invention.

In the foregoing compositions, one or more dissolution aids, buffering agents, coloring agents, flavoring agents, etc. commonly used in the field of the above preparations may also be suitably added as desired.

The term “pharmaceutical composition” refers to a solid or liquid composition in the form, concentration, and purity degree suitable for administration to a patient who, following administration, is induced to take place a desired physiological change, and the pharmaceutical composition is sterile and/or non-pyrogenic.

### Embodiment 1: an Analysis of the Ability of GM-080 to Inhibit Streptococcus Pyogenes NZ131

### (1) Inhibition Zone Experiment

An inhibition zone test was performed on pathogenic Streptococcus pyogenes clinical strain NZ131. The pathogenic Streptococcus pyogenes was post-activated and cultured for two days with TSA containing 5% defibrinated Sheep blood. Multiple lactic acid bacteria strains (as shown in Table 1) were cultured in a small amount with MRS culture medium. The lactic acid bacteria solution (adjusted to 1×10⁷ CFU/mL) was taken on the following day after activation to be activated for the second time, and was cultured overnight for use. Streptococci salivarius K12 and M18 were cultured and activated with BHI culture medium. After pathogenic bacteria NZ 131 were adjusted to an appropriate number (5×10⁸ CFU/mL), 100 μl of pathogenic bacteria NZ 131 was taken to be uniformly coated on a culture dish. After pathogenic bacteria NZ 131 was slightly dried, a hole was punched with a sterile glass tube of 11 mm. 100 μl of whole bacterial solution (lactic acid bacteria concentration was adjusted to 1×10⁹ CFU/ml) was added to each hole. After being placed at 37° C. and cultured for 48 hours, the size of the inhibition zone is measured by a vernier caliper. The size of the inhibition zone is the numerical value of the outer ring minus the inner ring, and the larger the numerical value, the stronger the bacteriostatic ability.

18 probiotic strains were screened with the inhibition zone experiment on Streptococcus pyogenes, and finally, it was found that Lactobacillus paracasei GM-080 had the best inhibitory effect (Table 1).

TABLE 1
A Comparison of the Ability of Different Bacteria
to Inhibit Streptococcus pyogenes NZ131

| | | inhibition zone (mm) |
|---|---|---|
| species | strains | mean ± SD |
| NC (MRS) | | 0.00 ± 0.25 |
| L. plantarum | #1 | 9.55 ± 1.95 |
| | #2 | 8.25 ± 0.11 |
| | #3 | 9.18 ± 0.98 |
| L. paracasei | GM-080 | 9.62 ± 0.43 |
| | #2 | 8.74 ± 0.46 |
| | #3 | 9.29 ± 1.61 |
| | #4 | 9.17 ± 0.94 |
| L. rhamnosus | #1 | 8.88 ± 1.24 |
| | #2 | 9.47 ± 0.74 |
| | #3 | 9.44 ± 0.82 |
| L. casei | #1 | 8.04 ± 0.38 |
| L. reuteri | #1 | 5.89 ± 1.12 |
| | #2 | 6.03 ± 1.03 |
| L. fermentum | #1 | 5.94 ± 0.29 |
| | #2 | 0.00 ± 0.49 |
| L. helveticus | #1 | 6.20 ± 0.79 |
| L. acdiophilus | #1 | 8.01 ± 0.04 |
| B. animalis | #1 | 0.00 ± 0.11 |

(2) An Efficacy Evaluation of the Lactic Acid Bacteria for Inhibiting the Attachment to Cells of Streptococcus pyogenes NZ131 in a Dish Coating Manner:

FaDu cells were seeded at 4×10⁵ cells/well in a 6-well culture dish, and after culturing overnight, the culture medium was replaced with a serum-free medium. Both lactic acid bacteria and pathogenic bacteria were adjusted to 4×10⁸ cfu/ml, and the pathogenic bacteria were mixed with lactic acid bacteria at 1:1. 100 μl of the mixed bacteria were taken in 6-well culture dish, which was placed at 37° C. and reacted for 2 hours, and after the culture medium was drawn off following the reaction, was washed three times with PBS. 0.5 mL of Trypsin/PBS was added to wipeout the cells, 5% of blood/TSA coating was performed after being diluted 10⁻¹, 10⁻², 10⁻³, 10⁻⁴, 10⁻⁵ times with PBS sequence. After being placed at 37° C. and cultured for 48 hours, the number of bacteria was calculated. Only the number of bacteria of the pure Streptococcus pyogenes infection group is set to 100%, and then the number of bacteria of other groups is converted to a relative number.

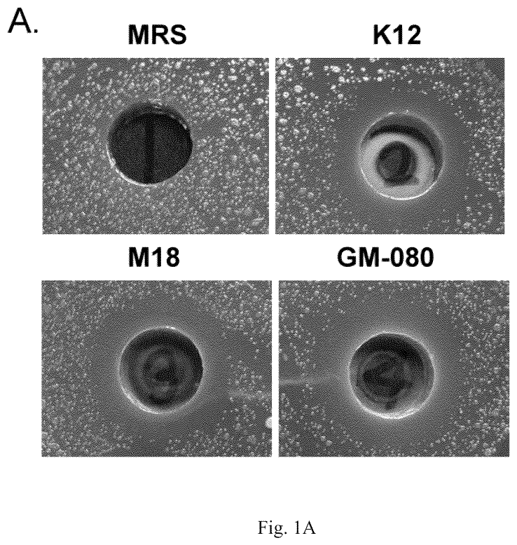

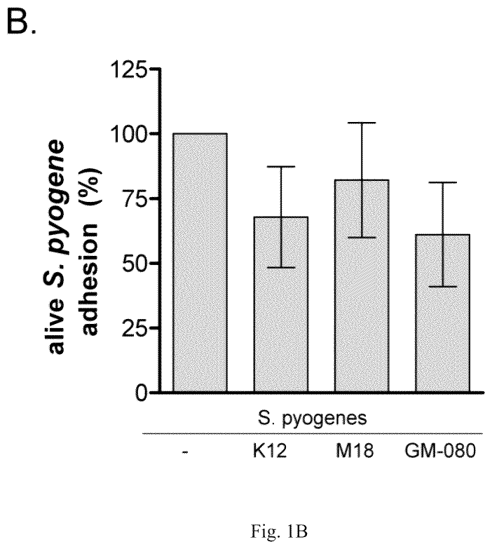

Among strains for inhibiting Streptococcus pyogenes found in the past, Streptococci salivarius K12 and M18 found in 2001 are famous strains. Thus, it is necessary to make a further comparison between the abilities of Lactobacillus paracasei GM-080 with Streptococci salivarius K12 and M18 to inhibit Streptococcus pyogenes. FIG. 1 is the analyses of the abilities to inhibit the growth of Streptococcus pyogenes and to inhibit the attachment to laryngopharyngeal cells. It was found from the results of FIG. 1 that the ability of GM-080 to inhibit Streptococcus pyogenes is slightly superior to Streptococci salivarius.

### Embodiment 2: A Test of Colonizing the Larynx of Probiotic Gel

1.26% of an agar gum aqueous solution was prepared. The agar gum aqueous solution, after boiling and dissolving, was allowed to stand at room temperature and to be cooled to 37° C. GM-080 viable bacteria powder was added, so that the final number of bacteria was 5×10¹⁰ CFU per gram of gel. The mixture is uniformly stirred, and it could be used after the agar gum was solidified into a solid state. Probiotic agar gum was placed in the stainless steel mesh lid of the mouse cage, instead of a drinking bottle to feed water, and an agar gum containing lactic acid bacteria was administrated to the mice continuously for 2 days. The face and the oral cavity of the mouse were disinfected with alcohol cotton, the wet sterile cotton rod was extended into the oral cavity of the mouse up to the pharynx, and the cotton rod was rotated three turns to collect the bacteria on the laryngopharynx. Immediately, applications directly on a MRS (Decne, Rogosa, and Sharpe) agar dish were performed with the cotton rod which had collected the specimens. Then, the agar dish was placed in an anaerobic incubator at 37° C. and cultured for 48 hours. The growth situation of lactic acid bacterial colonies was observed, lactic acid bacteria colonies were picked up for random amplification state deoxyribose nucleic acid (RAPD) analysis, and the ability of the strain to colonize larynx was evaluated. The DNA template concentration used for RAPD PCR was 10 ng, the RAPD primer used was LacP2 primer, that is, ACGCGCCCT (SEQ ID NO: 7). If the RAPD band produced by the PCR product was exactly the same as the standard one, it was regarded as the same strand.

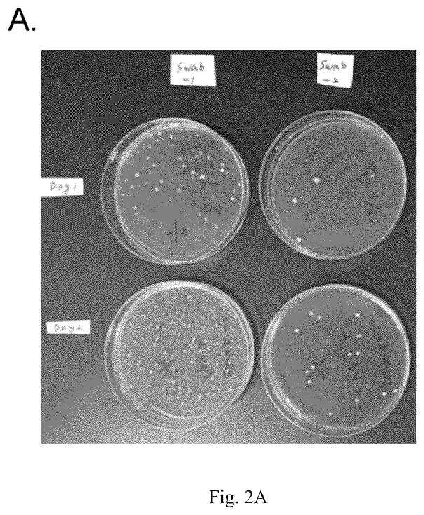

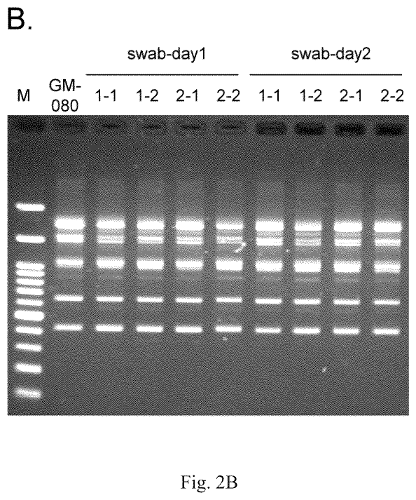

The ability of GM-080 to colonize the larynx was evaluated in mice, and the orally administered GM-080 gel was tested. FIG. 2 is an analysis of sites where GM-080 can colonize the laryngopharynx of a mouse in a viable bacteria form. It is proved from the results of FIG. 2 that there are viable GM-080 bacteria on the larynx in mouse larynx specimen viable bacteria by a RAPD meristematic method, which indicates that this strain is capable of colonizing the respiratory tract and achieving a better effect on inhibiting respiratory bacteria.

### Embodiment 3: an Analysis of Protection Efficacy of GM-080 of Preventing Streptococcus Pyogenes from Infecting

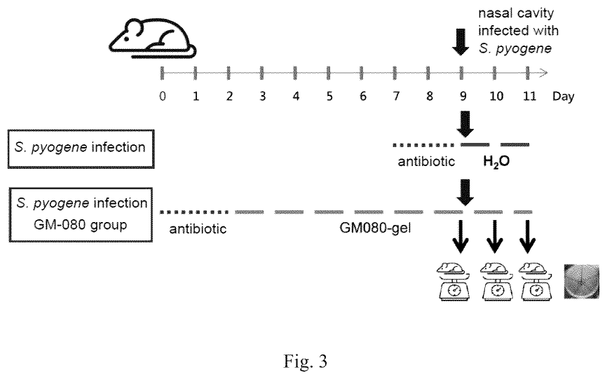

In case of an experimental group (4 mice per group), mice were fed with 0.3 mg/mL antibiotic enrofloxacin gels for two days before the experimental group was fed with probiotic GM-080 (5×10¹⁰ CFU/g) gels for 7 days. In case of a control group, mice was fed with 0.3 mg/mL antibiotic enrofloxacin gels for two days before the mice was directly infected with pathogenic bacteria. Streptococcus pyogenes 2×10⁹ cfu/0.04 mL/one nasal cavity was administered as an intranasal instillation on day 8 (to achieve bilateral nasal infections). The changes in body weights of the mice within 2 days before infection and after infection were observed and pharyngeal tissues were taken out to evaluate the number of Streptococcus pyogenes therein, and the experimental procedure is shown in FIG. 3.

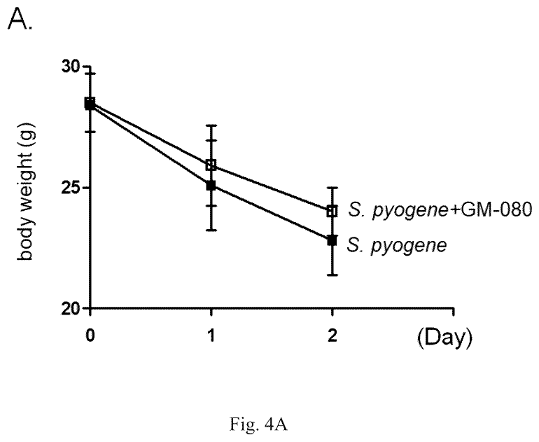

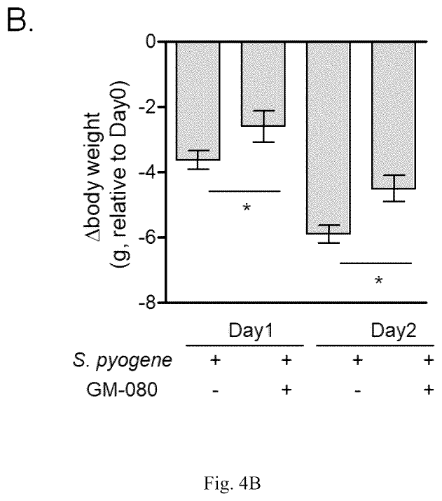

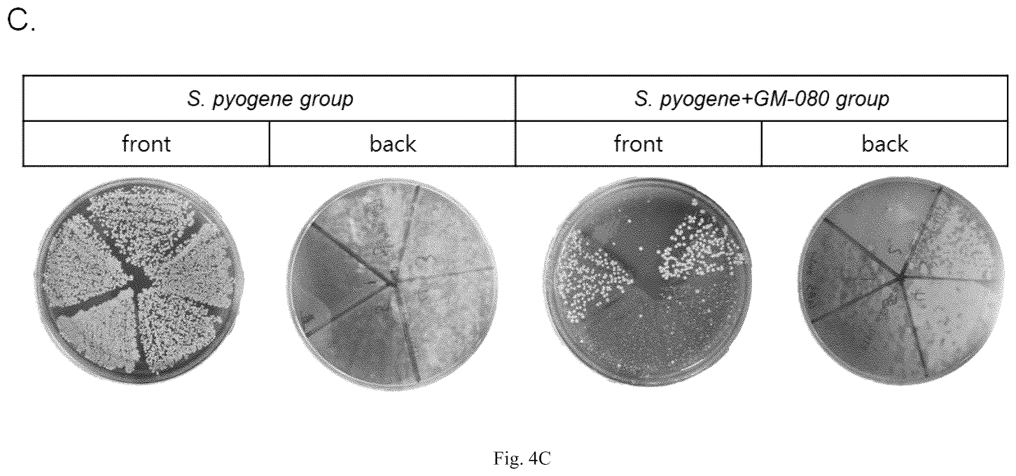

A mode of Streptococcus pyogenes infection in a mouse is further established, in which at first the flora originally on laryngopharynx are removed with an antibiotic gel, and then the laryngopharynx is infected with Streptococcus pyogenes. FIG. 4 is the analyses of the mitigation of the reduction in weight of mice caused by Streptococcus pyogenes by GM-080 and the number of germs on the larynx. It is found from the results of FIG. 4 that the reduction in weight of mice is caused by the infection of the larynx of mice with Streptococcus pyogenes. However, it is found from the results of FIGS. 4A and B that the reduction in weight of mice caused by Streptococcus pyogenes could be mitigated under the oral administration of GM-080 gel. In addition, it is found from the culture of pharyngeal specimens of FIG. 4C that the number of viable bacteria of the local Streptococcus pyogenes could be indeed effectively reduced by administering GM-080 gel.

### Embodiment 4: Predicting an Antimicrobial Peptide of Whole Gene Desequencing of GM-080

Whole gene desequencing of strain GM-080 is completed by using 2nd and 3rd generation sequencing. The subsequent predictive analysis of the assembled gene sequences is performed at a website of Antimicrobial peptides (AMPs) (AMP: https://awi.cuhk.edu.cn/dbAMP) to identify possible antimicrobial peptides. The true presence of the antimicrobial peptide in the genome of lactic acid bacteria is tested by fluorescent quantitative PCR meristematic method, with the DNA template concentration used in fluorescent quantitative PCR (Q-PCR) being 10 ng. The primers of AMP-01026 used for Q-PCR include a Forward primer which is GGCCGTGTAGGATTCGGATAA (SEQ ID NO:3) and a Reverse primer which is GTGGTAAACGAAAGTGCCCC (SEQ ID NO:4), and product size is 119 bp. The primers of AMP-01240 used for Q-PCR include a Forward primer which is AGATGCGAGCGAAAACTCGT (SEQ ID NO:5) and a Reverse primer which is ATTGGAGGTGACAGCATCCG (SEQ ID NO:6), and product size is 116 bp.

TABLE 2
Primer Sequences of AMP-01026 and AMP-01240 used for Q-PCR

| Name of Primer | Sequence Number | Nucleotide Sequence |
|---|---|---|
| AMP-01026 Forward Primer | SEQ ID NO: 3 | GGCCGTGTAGGATTCGGATAA |
| AMP-01026 Reverse Primer | SEQ ID NO: 4 | GTGGTAAACGAAAGTGCCCC |
| AMP-01240 Forward Primer | SEQ ID NO: 5 | AGATGCGAGCGAAAACTCGT |
| AMP-01240 Reverse Primer | SEQ ID NO: 6 | ATTGGAGGTGACAGCATCCG |
| LacP2 Primer | SEQ ID NO: 7 | ACGCGCCCT |

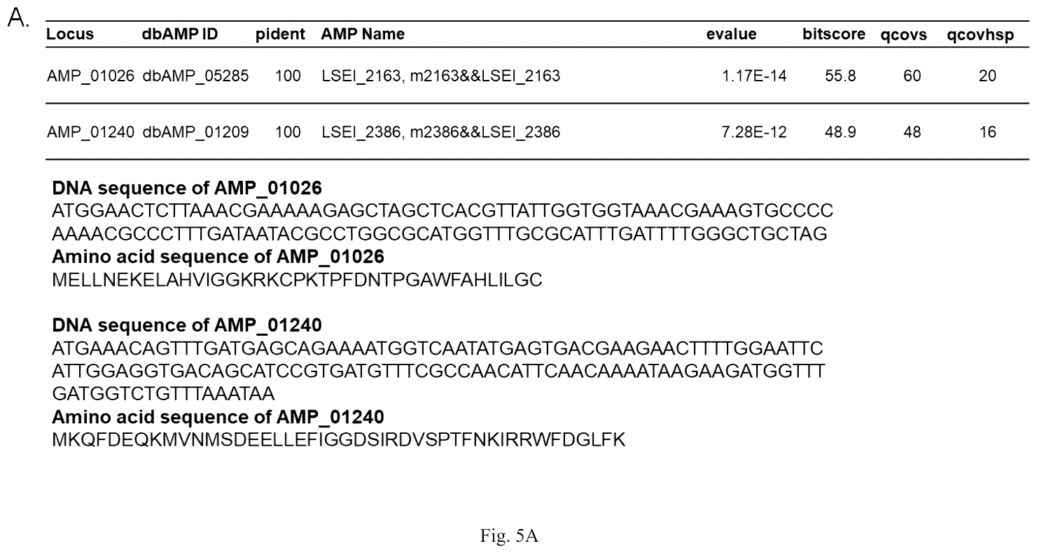

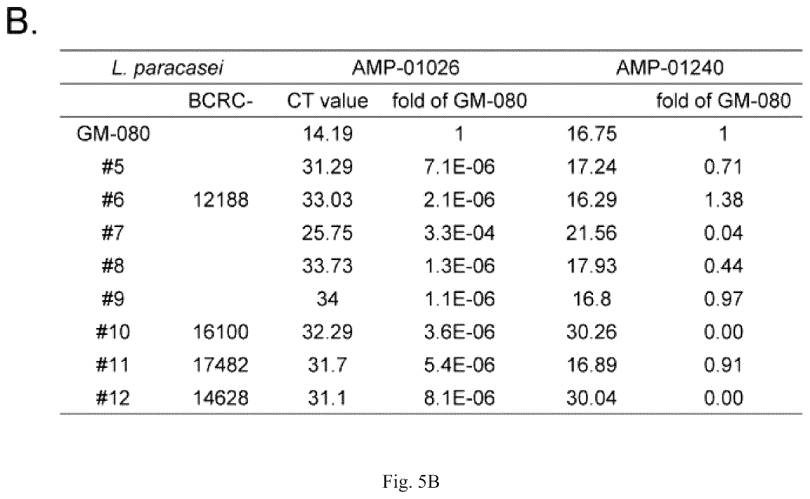

FIG. 5 is the analyses of the DNA and amino acid sequences of antimicrobial peptides AMP-01026 and AMP-01240 of GM-080, and of differences of expression amount thereof from other Lactobacilli paracasei measured by Q-PCR. It is found from the results of FIG. 5B that the two segments of antimicrobial peptide of GM-080 do have a larger amount of expression than other Lactobacillus paracasei, and especially the expression amount of AMP-01026 is much more than other Lactobacillus paracasei.

### Embodiment 5: Confirmation of Bacteriostatic Effect of Antimicrobial Peptide

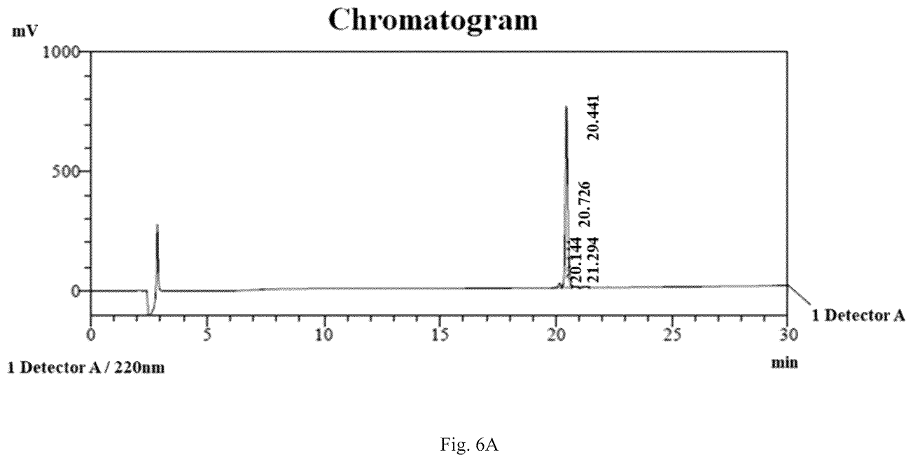

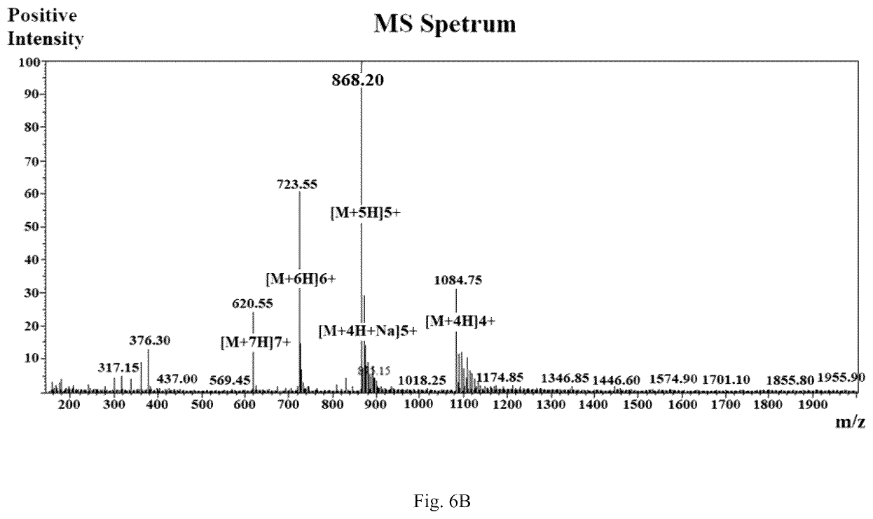

Mission Company was entrusted to perform the synthesis of the two segments of antimicrobial peptide to obtain the powder of antimicrobial peptide, the two segments of antimicrobial peptide including AMP-01026 set forth as MELLNEKELAHVIGGKRKCPKTPFDNTPGAWFAHLILGC (SEQ ID NO: 1) with the purity and analytical report as shown in FIG. 6 and AMP-01240 set forth as

(SEQ ID NO: 2)
MKQFDEQKMVNMSDEELLEFIGGDSIRDVSPTFNKIRRWFDGLFK

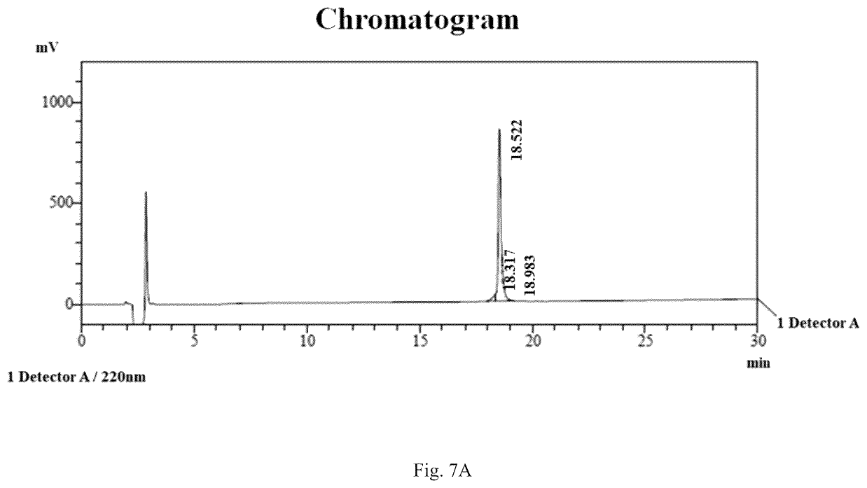

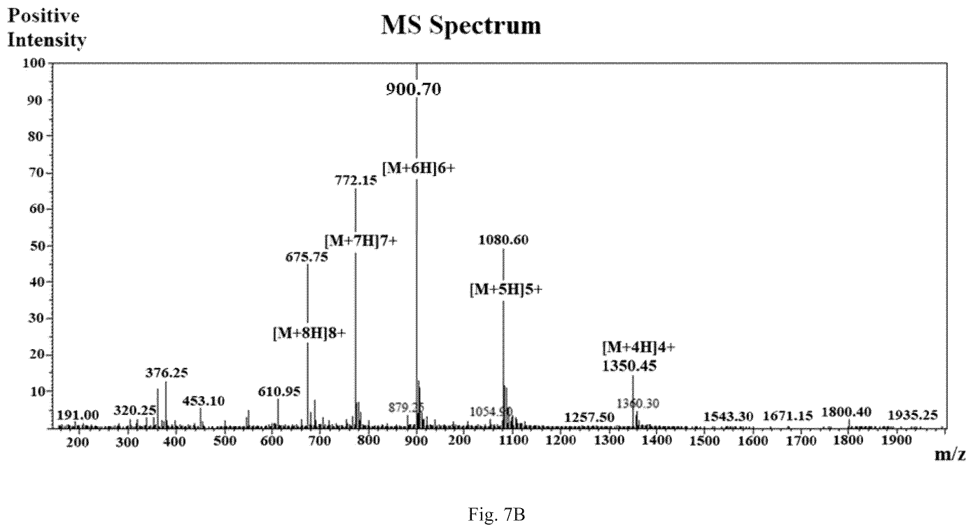

with the purity and analytical report as shown in FIG. 7. The powder was diluted with DMSO to different concentrations. Subsequently, after being co-cultured with pathogenic streptococcus for different time periods, it was confirmed whether the antimicrobial peptides were the antimicrobial peptide of GM-080 capable of inhibiting Streptococcus pyogenes NZ131 by OD6oonm or in a dish coating manner simultaneously.

TABLE 3
Peptides AMP-01026 and AMP-01240 Sequences and Molecular Weight

| Name of | Sequence | | Molecular |
|---|---|---|---|
| Peptide | Number | Amino Acid Sequence | Weight |
| AMP-01026 | SEQ ID NO: 1 | MELLNEKELAHVIGGKRKCPKTPFDNTPGAWFAHLILGC | 4336.13 |
| AMP-01240 | SEQ ID NO: 2 | MKQFDEQKMVNMSDEELLEFIGGDSIRDVSPTFNKIRRWFDGLFK | 5399.18 |

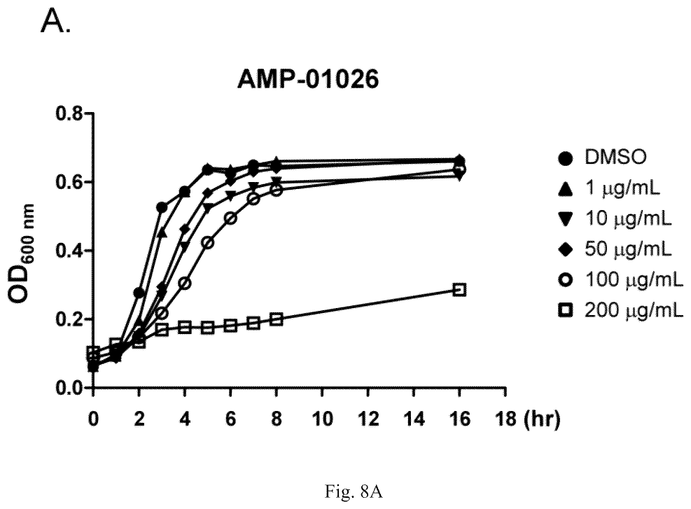

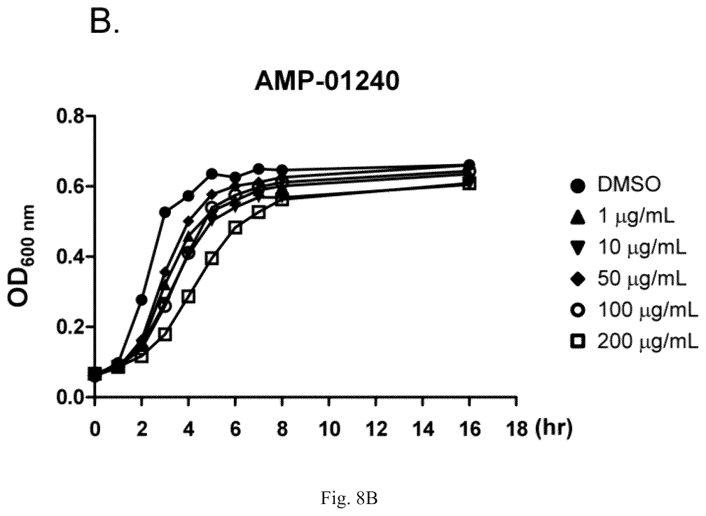

FIG. 8 is analyses for evaluating the abilities of AMP-01026 and AMP-01240 to inhibit Streptococcus pyogenes in a co-culture manner. It is found from the results of FIG. 8A that AMP-01026 (SEQ ID NO: 1) in different concentrations (10-200 mg/mL) already had a good inhibition effect of Streptococcus pyogenes from the time that it had been co-cultured for 2-3 hours.

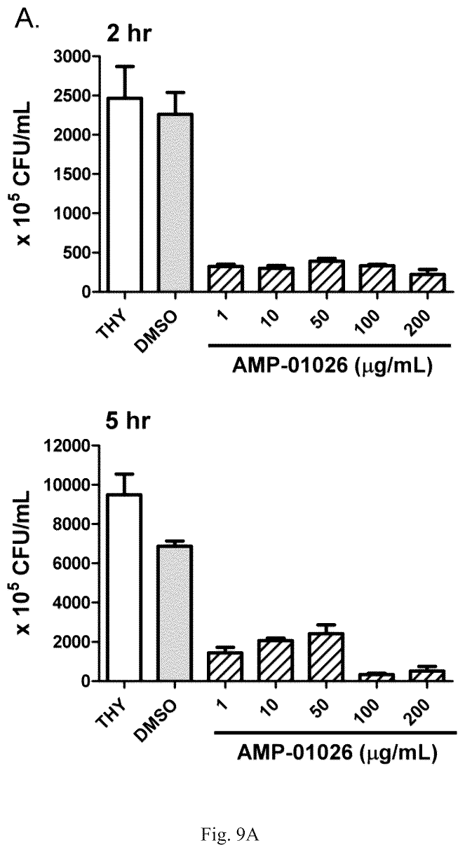

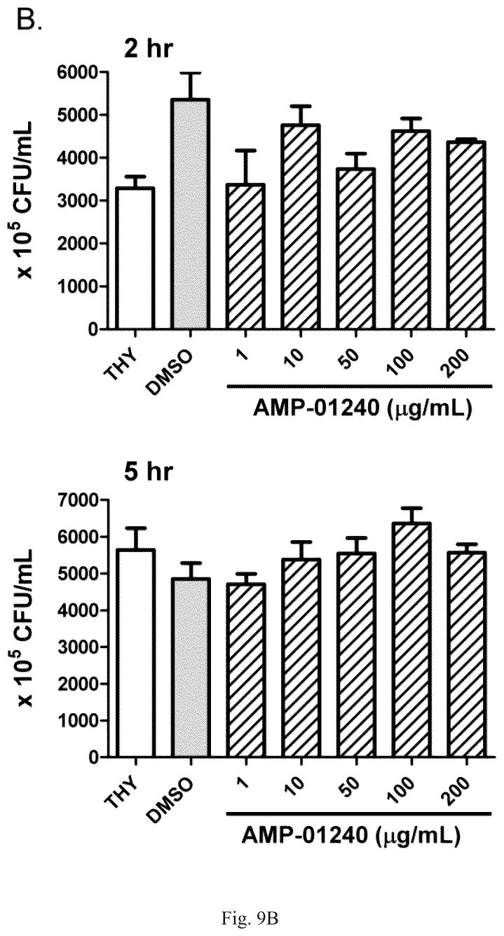

FIG. 9 is an evaluation of the abilities of AMP-01026 and AMP-01240 to inhibit Streptococcus pyogenes in a dish coating manner, and the number of viable bacteria of pathogenic bacteria is further confirmed in a dish coating manner. It is found from the results of FIG. 9A that AMP-01026 in different concentrations (1-200 mg/mL) could significantly inhibit the growth of Streptococcus pyogenes at the time that it had been co-cultured for 2 or 5 hours, indicating that antimicrobial peptide AMP-01026 produced in large amounts by GM-080 has the ability to inhibit the growth of Streptococcus pyogenes.

It is found from FIGS. 8B and 9B that another antimicrobial peptide segment AMP-01240 (SEQ ID NO: 2) in different concentrations (1-200 mg/mL) is not capable of inhibiting the growth of Streptococcus pyogenes.

The results show that an antimicrobial peptide of GM-080 is selective for different pathogenic bacteria, and whether it has a bacteriostatic ability needs to be confirmed by experiment.

It could be seen by summarizing above results that:

1. In the inhibition zone experiment on Streptococcus pyogenes, Lactobacillus paracasei GM-080 has the best effect on inhibiting Streptococcus pyogenes compared with 18 probiotic strains.

2. Lactobacillus paracasei GM-080 is capable of colonizing the respiratory tract and achieving a better effect on inhibiting respiratory bacteria.

3. The number of viable bacteria of local Streptococcus pyogenes can be effectively reduced by administrating GM-080 gel.

4. In addition, the peptides comprising the sequence set forth in SEQ ID NO: 1 provided in the present invention also have the efficacy of resisting to Streptococcus pyogenes.

Accordingly, the Lactobacillus paracasei GM-080 and antimicrobial peptide thereof provided in the present invention is highly suitable for the development and preparation of compositions for resisting Streptococcus pyogenes, aiding in the development of commercial products and industry for prevention of upper respiratory diseases.

According to the content disclosed in the preferred embodiments of the present specification, it will be apparent to those skilled in the art that the foregoing embodiments are illustrations only. The present invention can be implemented by one skilled in the art by many conversions and substitutions without departing from the technical features of the present invention. There are various variations of the present invention and the implementations thereof are not impeded according to the embodiments described in the present specification. The scope of the invention is defined in the claims provided herein and encompasses the foregoing methods and structures and the equivalents thereto.

The above-mentioned multiple efficacies sufficiently conform to the patentability of inventions including novelty and inventive step, therefore, the patent application is filed under the patent law, and sincerely requests to approve this invention patent application to encourage inventions.

### [Biological Material Deposit]

Lactobacillus paracasei (also known as Lacticaseibacillus paracasei) GM-080, deposited in the Food Industry Development Research Institute under the deposit number BCRC 910220 and deposited in the China Center for Type Culture Collection (CCTCC) under the deposit number CCTCC M 204012.

1. A peptide with an antimicrobial effect, the peptide sequence is set forth in SEQ ID NO: 1.

2. The peptide as claimed in claim 1, being secreted by Lactobacillus paracasei GM-080 having a deposit number of CCTCC M 204012.

3. A composition with an antimicrobial effect, comprising the peptide as claimed in claim 1.

4. The composition as claimed in claim 3, wherein the composition is capable of further comprising a pharmaceutically acceptable carrier.

5. The composition as claimed in claim 3, wherein the composition is in the form of a solution, a suspension, an emulsion, a powder, a pastille, a pill, a syrup, a buccal ingot, a tablet, a chewing gum, a thick slurry, or a capsule.

6. The composition as claimed in claim 3, wherein the composition is capable of further comprising an edible material.

7. A method of Lactobacillus paracasei GM-080 or peptides for preparing an antimicrobial composition, the Lactobacillus paracasei having a deposit number of CCTCC M 204012, the antimicrobial effect being intended to inhibit the growth of microorganisms, the peptide sequence being set forth in SEQ ID NO: 1.

8. The method as claimed in claim 7, wherein the microorganism is Streptococcus.

9. The method as claimed in claim 7, the microorganism is Streptococcus pyogenes.

10. The method as claimed in claim 7, wherein the composition is a pharmaceutical composition, a nutritional supplement, or a health care food.

11. The method as claimed in claim 7, wherein the peptide is secreted by Lactobacillus paracasei GM-080.
